(19) United States
(12) Patent Application Publication
(10) Pub. No.: US 2023/0372903 A1
(43) Pub. Date: Nov. 23, 2023

(54) CATALYSTS FOR SELECTIVE OXIDATION OF METHANOL TO DIMETHOXYMETHANE AND RELATED METHODS

(71) Applicant: King Abdullah University of Science and Technology, Thuwal (SA)

(72) Inventors: Kuo-Wei HUANG, Thuwal (SA); Ding-Jier YUAN, Thuwal (SA)

(21) Appl. No.: 17/914,503

(22) PCT Filed: Mar. 26, 2021
(86) PCT No.: PCT/IB2021/052536

§ 371 Date: Sep. 26, 2022

Related U.S. Application Data
(60) Provisional application No. 62/994,906, filed on Mar. 26, 2020.

(51) Int. Cl. B01J 23/22; B01J 37/02; B01J 23/10; B01J 21/02; C07C 41/50

(52) U.S. Cl. CPC B01J 23/22; B01J 37/0201; B01J 23/10; B01J 21/02; C07C 41/50

(57) ABSTRACT

Embodiments include catalyst compositions and methods of synthesizing catalyst compositions for the selective oxidation of methanol to dimethoxymethane, as well as methods of selective oxidation of methanol to dimethoxymethane using catalyst compositions. The catalyst composition can comprise vanadium oxide and a mixed metal oxide, wherein the vanadium oxide is supported on the mixed metal oxide and wherein the mixed metal oxide includes a redox component and an acid component. The method of selective oxidation of methanol to dimethoxymethane can comprise at least the following step: contacting methanol with a catalyst composition in the presence of an oxidizing agent to produce dimethoxymethane.

### BACKGROUND

Dimethoxymethane (DMM) is a high-value chemical widely used in industry. For example, DMM can be used as a building block in organic synthesis. Due to its low toxicity, DMM can also be used as an environmentally friendly solvent in various applications which include pharmaceutical and organic synthesis. Due to its high oxygen content and chemical stability, DMM can further be used as a fuel additive. Conventional methods of producing dimethoxymethane involve a two-step oxidation process: (1) oxidation of methanol to formaldehyde followed by (2) a dehydration reaction between methanol and formaldehyde via acidic catalysis. Such methods, however, are known to have high energy costs, cause substantial damage to equipment (e.g., via corrosion), and result in undesirable complex side reactions.

### SUMMARY

In some aspects, the present invention provides a catalyst composition for selective oxidation of methanol to dimethoxymethane, optionally in one step. A catalyst composition for use in the formation of dimethoxymethane by selective oxidation of methanol may include a mixed metal oxide support impregnated with a vanadium oxide, wherein the mixed metal oxide support includes at least a redox component and an acid component.

In further aspects, the present invention provides a method of selective oxidation of methanol to dimethoxymethane in one step. The method may include one or more of the following steps: (a) activating a precatalyst to obtain a catalyst; (b) contacting methanol with the catalyst in the presence of an oxidizing agent to produce dimethoxymethane; (c) separating the catalyst from at least the reaction products to recover the catalyst; and (d) repeating step (b) one or more times with the recovered catalyst.

In additional aspects, the present invention provides a method of synthesizing a catalyst composition for selective oxidation of methanol to dimethoxymethane. The method may include one or more of the following steps: (a) forming a mixed metal oxide support by coprecipitation; (b) treating the mixed metal oxide support; (c) impregnating the mixed metal oxide support with a vanadium oxide; and (d) treating the mixed metal oxide support to obtain a catalyst composition.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a schematic diagram of a reaction scheme for the selective oxidation of methanol to produce dimethoxymethane, in accordance with one or more embodiments of the present invention.

FIG. 2 is a flowchart of a method of forming dimethoxymethane by selective oxidation of methanol, in accordance with one or more embodiments of the present invention.

FIG. 3 is a flowchart of a method of synthesizing catalyst compositions for selective oxidation of methanol to dimethoxymethane in one step, according to one or more embodiments of the invention.

FIG. 4 is a schematic diagram of a Ce—Al mixed metal oxides supported vanadium catalyst for selective oxidation of methanol to dimethoxymethane, in accordance with one or more embodiments of the present invention.

FIG. 5 is a graphical view illustrating the effect of Ce/Al ratios on conversion and selectivity, in accordance with one or more embodiments of the present invention.

FIG. 6 is a N₂ sorption isotherm of a catalyst composition, according to one or more embodiments of the invention.

FIG. 7 is a graphical view of a pore size distribution of catalyst compositions, according to one or more embodiments of the invention.

FIG. 8 is a graphical view of a wide-angle XRD pattern of catalyst compositions, according to one or more embodiments of the invention.

FIGS. 9A-9E are HR-TEM images of the following catalyst compositions: a) V₂O₅/Ce₁Al₉Oₓ; b) V₂O₅/Ce3Al7Oₓ; c) V₂O₅/CeAlOₓ; d) V₂O₅/Ce₇Al₃Oₓ; e) V₂O₅/Ce₉Al₁Oₓ, according to one or more embodiments of the invention.

FIGS. 10A-10B are graphical views of (A) NH₃-TPD and (B) NH₃ uptake of catalyst compositions, according to one or more embodiments of the invention.

FIG. 11 is a graphical view of a H₂-TPR of catalyst compositions, according to one or more embodiments of the invention.

FIG. 12 is Raman spectra of catalyst compositions, according to one or more embodiments of the invention.

FIGS. 13A-13B are graphical views of X-ray adsorption of Ce (A) Ce3d XPS and (B) Ce L3-edge XANES of catalyst compositions, according to one or more embodiments of the invention.

FIGS. 14A-14B are XPS peak fittings for (A) V2p and (B) O1s of catalyst compositions, according to one or more embodiments of the invention.

FIG. 15 are EPR spectra of catalyst compositions, according to one or more embodiments of the invention.

FIG. 16 is a graphic view illustrating the stability of a V₂O₅/CeAlOₓ catalyst composition, in accordance with one or more embodiments of the present invention.

FIGS. 17A-17B is a graphical view characterizing a spent catalyst with (A) TG analysis and (B) Raman spectra of a spent catalyst composition, according to one or more embodiments of the invention.

### DETAILED DESCRIPTION

### Definitions

The terms recited below have been defined as described below. All other terms and phrases in this disclosure shall be construed according to their ordinary meaning as understood by one of skill in the art.

As used herein, “catalyst composition(s)” refers to catalysts and pre-catalysts. A “catalyst” generally refers to a material that is catalytically active in a reaction. A “pre-catalyst” generally refers to a material that is catalytically inactive in a reaction and that is capable of becoming catalytically active (e.g., capable of becoming a catalyst) upon being activated. The term “catalyst composition” includes heterogeneous catalysts. The term “heterogeneous catalyst” refers to a catalyst having a phase which is different from the phase of reactants and/or products.

As used herein, the term “atomic ratio” of an atom i is calculated according to the following equation: Atomic Ratio=Nᵢ/(N_{Total}), where Nᵢ is the number of atoms i and N_{Total} is the total number of atoms. The atomic ratio may also be calculated according to the following equation: Atomic Ratio=Nᵢ/Nⱼ where Nᵢ is the number of atoms of component i and Nⱼ is the number of atoms of component j. For example, the atomic ratio of Ce/Al for a mixed metal oxide support having the formula CeAlO_{z} is 1:1, where the number of Ce atoms is 1 and the number of Al atoms is 1.

As used herein, the term “weight hourly space velocity” (WHSV) refers to the weight of feed flowing per unit weight of the catalyst per hour.

As used herein, the term “conversion” refers to the percentage of a reactant which reacts to form a product. For example, methanol conversion can refer to the percentage of methanol which reacts to form dimethoxymethane.

As used herein, the term “selectivity” refers to the percentage of a reacted reagent which is converted to a product. For example, selectivity can refer to the percentage of reacted methanol which is converted to dimethoxymethane.

As used herein, the terms “vanadium oxide” and “vanadia” refer to compounds of the formula VₓO_{y}, where x>0 and y is >0. The term includes mixtures of at least two vanadium oxides. Examples of vanadium oxides include, without limitation, VO, VO₂, VO₄³⁻, V₂O₃, V₂O₄, V₂O₅, and the like. Vanadium can be present in any oxidation state. For example, vanadium can be present as V₅₊, V₄₊, V₃₊, V₂₊, or any combination thereof.

As used herein, the terms “cerium oxide” and “ceria” refer to compounds of the formula CeₓO_{y}, where x>0 and y is >0. The term includes mixtures of at least two cerium oxides. Examples of cerium oxides include, without limitation, CeO₂, Ce₂O₃, and the like. Cerium can be present in any oxidation state. For example, cerium can be present as Ce³⁺, Ce⁴⁺, or any combination thereof.

As used herein, the term “oxide” refers to any compound including oxygen. Examples of oxides include, without limitation, metal oxides (e.g., MₓO_{y}), metal oxyhalides (e.g., MₓO_{y}X_{z}, where X is independently, at each occurrence, fluoro, bromo, chloro, or iodo), and metal oxy-hydroxides (e.g., MₓO_{y}(OH)_{z}). Additional examples of oxides include metal oxynitrates (e.g., MₓO_{y}(NO₃)_{z}), metal phosphates (e.g., Mₓ(PO₄)_{y}), metal oxycarbonates (MₓO_{y}(CO₃)_{z}), metal carbonates, and the like, where x, y, and z are each independently at least 1. In addition, the term also includes any of the foregoing oxides in which the metal is replaced by a metalloid.

As used herein, the term “mixed metal oxide” refers to a compound including two or more metals or metalloids and oxygen. For example, the term includes compounds of the formula: M¹ₓM²_{y}O_{z}, where M¹ is a first metal or metalloid, M² is a second metal or metalloid, O is oxygen, x is from 1 to 100, y is from 1 to 100, and z is from 1 to 100. In another example, the term includes compounds of the formula: M¹ₓO_{z1}-M²_{y}O_{z2}, where M¹ is a first metal or metalloid, M² is a second metal or metalloid, O is oxygen, x is from 1 to 100, y is from 1 to 100, z1 is from 1 to 100, and z2 is from 1 to 100. M¹ and M² can be the same or different (e.g., in terms of metal or metalloid and/or oxidation states). M¹ and M² can include the same metal or metalloid and have different oxidation states. M¹ and M² can include different metals or metalloids and have the same oxidation states.

As used herein, the term “oxidizing agent” refers to a substance that is able to bring about oxidation in another substance. Examples of oxidizing agents include, without limitation, oxygen sources, such as air, pure O₂, or any other substance including oxygen.

### DISCUSSION

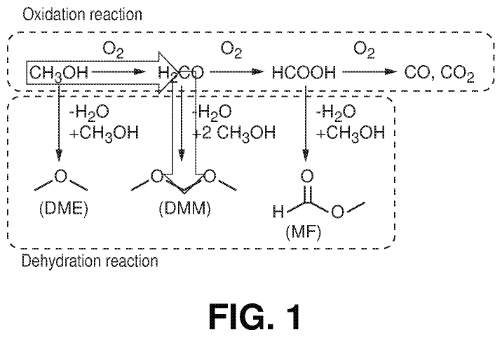

The present invention provides catalyst compositions for the highly selective oxidation of methanol to dimethoxymethane. For example, in some embodiments, the selective oxidation of methanol to dimethoxymethane can proceed directly in one step according to the reaction scheme shown in FIG. 1. As illustrated in FIG. 1, the catalyst compositions can promote formation of dimethoxymethane over other reaction products, such as dehydration product dimethyl ether and over-oxidation products such as carbon dioxide and methyl formate. While not wishing to be bound to a theory, it is believed that the high selectivity towards dimethoxymethane can be attributed to the presence of redox sites on the catalyst composition which promote oxidation of methanol to formaldehyde and acid sites on the catalyst composition which promote dehydration of formaldehyde to dimethoxymethane. In other words, the redox site of the catalyst composition can facilitate the oxidation of methanol to formaldehyde and the acid site can make it easier for the dehydration reaction between the methanol and formaldehyde to occur, improving catalytic performance.

Accordingly, the catalyst compositions of the present invention can thus provide at least one of the following advantages over conventional catalysts: (a) the catalyst compositions can be used to produce dimethoxymethane in a one-step catalytic selective oxidation process; (b) the catalyst compositions can achieve high methanol conversions; (c) the catalyst compositions can exhibit high selectivity for dimethoxymethane; (d) the catalyst compositions can be highly stable with long lifetimes (e.g., >500 hr); (e) the catalyst compositions can be fabricated using strategies that permit modulation of the atomic ratio of redox and acid components to promote formation of desired products; and (f) the catalyst compositions can include heterogeneous catalysts thereby permitting recovery and reuse of the catalyst compositions one or more times. These and other advantages of the catalyst compositions are described herein.

### Catalyst Compositions

In one or more aspects, the present invention provides catalyst compositions comprising vanadium oxide and a mixed metal oxide (e.g., a mixed metal oxide support). In some embodiments, the vanadium oxide is supported on the mixed metal oxide. In some embodiments, the mixed metal oxide (e.g., the mixed metal oxide support) is impregnated with the vanadium oxide. For example, in some embodiments, the mixed metal oxide support includes vanadium oxide dispersed within and/or throughout the pores of the mixed metal oxide support. In some embodiments, the mixed metal oxide includes a redox component (e.g., providing redox sites for the oxidation of methanol to formaldehyde) and an acid component (e.g., provide acid sites for the dehydration reaction between formaldehyde and methanol). In some embodiments, the redox component includes cerium. For example, in some embodiments, the mixed metal oxide includes cerium and an acid component. In some embodiments, the catalyst composition is a heterogeneous catalyst. For example, in some embodiments, the catalyst is a solid heterogeneous catalyst.

The vanadium oxide can provide the catalytic active sites of the catalyst composition. For example, in some embodiments, the active sites include vanadia sites. In some embodiments, the vanadium oxide is represented by the chemical formula: VₘOₙ, where m is at least 1 and n is at least 1. In some embodiments, the vanadium oxide includes V₂O₅. In some embodiments, the vanadium oxide includes VO. In some embodiments, the vanadium oxide includes VO₂. In some embodiments, the vanadium oxide includes VO₄³⁻. In some embodiments, the vanadium oxide includes V₂O₃. In some embodiments, the vanadium oxide includes V₂O₄. In some embodiments, the vanadium oxide includes at least one of the following: V₂O₅, VO, VO₂, VO₄³⁻, V₂O₃, and V₂O₄.

The mixed metal oxide can provide redox sites for the oxidation of methanol to formaldehyde. For example, in some embodiments, the mixed metal oxide includes a redox component. In some embodiments, the redox component includes a cerium component. In some embodiments, the redox sites include ceria sites. The cerium component can include cerium in any oxidation state, cerium in elemental form, cerium oxides, or any combination thereof. In some embodiments, the cerium component includes elemental cerium. In some embodiments, the cerium component includes Ce²⁺. In some embodiments, the cerium component includes Ce³⁺. In some embodiments, the cerium component includes CeO₂. In some embodiments, the cerium component includes Ce₂O₃. In some embodiments, the cerium component includes at least one of the following: Ce, Ce²⁺, Ce³⁺, CeO₂, and Ce₂O₃.

The mixed metal oxide can provide acid sites for the dehydration reaction between formaldehyde and methanol. For example, in some embodiments, the mixed metal oxide includes an acid component. The acid component can include a metal, a metalloid, or any combination thereof. For example, the acid component can include transition metals, post-transition metals, lanthanides, actinides, metalloids, and combinations thereof. Examples of acid components include, without limitation, aluminum (Al), chromium (Cr), cobalt (Co), silicon (Si), iron (Fe), manganese (Mn), molybdenum (Mo), niobium (Nb), antimony (Sb), scandium (Sc), yttrium (Y), lanthanum (La), titanium (Ti), zirconium (Zr), hafnium (Hf), tantalum (Ta), tungsten (W), rhenium (Re), ruthenium (Ru), rhodium (Rh), iridium (Ir), nickel (Ni), palladium (Pd), platinum (Pt), copper (Cu), silver (Ag), gold (Au), zinc (Zn), boron (B), gallium (Ga), indium (In), lead (Pb), phosphorus (P), arsenic (As), bismuth (Bi), praseodymium (Pr), neodymium (Nd), samarium (Sm), and terbium (Tb).

In some embodiments, the mixed metal oxide can be represented by the following chemical formula: CeₓM_{y}O_{z}, where M is an acid component, x is greater than or at least 1, y is greater than 0 or at least 1, and z is greater than 0 or at least 1. For example, in some embodiments, the mixed metal oxide includes: CeₓAl_{y}O_{z}, wherein x, y, and z are as defined above. In some embodiments, the mixed metal oxide includes CeₓCr_{y}O_{z}, wherein x, y, and z are as defined above. In some embodiments, the mixed metal oxide includes CeₓCo_{y}O_{z}, wherein x, y, and z are as defined above. In some embodiments, the mixed metal oxide includes CeₓSi_{y}O_{z}, wherein x, y, and z are as defined above. In some embodiments, the mixed metal oxide includes CeₓFe_{y}O_{z}, wherein x, y, and z are as defined above. In some embodiments, the mixed metal oxide includes CeₓMn_{y}O_{z}, wherein x, y, and z are as defined above. In some embodiments, the mixed metal oxide includes CeₓMo_{y}O_{z}, wherein x, y, and z are as defined above. In some embodiments, the mixed metal oxide includes CeₓNb_{y}O_{z}, wherein x, y, and z are as defined above. In some embodiments, the mixed metal oxide includes CeₓSb_{y}O_{z}, wherein x, y, and z are as defined above. In some embodiments, the mixed metal oxide includes CeₓSc_{y}O_{z}, wherein x, y, and z are as defined above. In some embodiments, the mixed metal oxide includes CeₓY_{y}O_{z}, wherein x, y, and z are as defined above. In some embodiments, the mixed metal oxide includes CeₓLa_{y}O_{z}, wherein x, y, and z are as defined above. In some embodiments, the mixed metal oxide includes CeₓTi_{y}O_{z}, wherein x, y, and z are as defined above. In some embodiments, the mixed metal oxide includes CeₓZr_{y}O_{z}, wherein x, y, and z are as defined above. In some embodiments, the mixed metal oxide includes CeₓHf_{y}O_{z}, wherein x, y, and z are as defined above. In some embodiments, the mixed metal oxide includes CeₓTa_{y}O_{z}, wherein x, y, and z are as defined above. In some embodiments, the mixed metal oxide includes CeₓW_{y}O_{z}, wherein x, y, and z are as defined above. In some embodiments, the mixed metal oxide includes CeₓRe_{y}O_{z}, wherein x, y, and z are as defined above. In some embodiments, the mixed metal oxide includes CeₓRu_{y}O_{z}, wherein x, y, and z are as defined above. In some embodiments, the mixed metal oxide includes CeₓRh_{y}O_{z}, wherein x, y, and z are as defined above. In some embodiments, the mixed metal oxide includes CeₓIr_{y}O_{z}, wherein x, y, and z are as defined above. In some embodiments, the mixed metal oxide includes CeₓNi_{y}O_{z}, wherein x, y, and z are as defined above. In some embodiments, the mixed metal oxide includes CeₓPd_{y}O_{z}, wherein x, y, and z are as defined above. In some embodiments, the mixed metal oxide includes CeₓPt_{y}O_{z}, wherein x, y, and z are as defined above. In some embodiments, the mixed metal oxide includes CeₓCu_{y}O_{z}, wherein x, y, and z are as defined above. In some embodiments, the mixed metal oxide includes CeₓAg_{y}O_{z}, wherein x, y, and z are as defined above. In some embodiments, the mixed metal oxide includes CeₓAu_{y}O_{z}, wherein x, y, and z are as defined above. In some embodiments, the mixed metal oxide includes CeₓZn_{y}O_{z}, wherein x, y, and z are as defined above. In some embodiments, the mixed metal oxide includes CeₓB_{y}O_{z}, wherein x, y, and z are as defined above. In some embodiments, the mixed metal oxide includes CeₓGa_{y}O_{z}, wherein x, y, and z are as defined above. In some embodiments, the mixed metal oxide includes CeₓIn_{y}O_{z}, wherein x, y, and z are as defined above. In some embodiments, the mixed metal oxide includes CeₓPb_{y}O_{z}, wherein x, y, and z are as defined above. In some embodiments, the mixed metal oxide includes CeₓP_{y}O_{z}, wherein x, y, and z are as defined above. In some embodiments, the mixed metal oxide includes CeₓAs_{y}O_{z}, wherein x, y, and z are as defined above. In some embodiments, the mixed metal oxide includes CeₓBi_{y}O_{z}, wherein x, y, and z are as defined above. In some embodiments, the mixed metal oxide includes CeₓPr_{y}O_{z}, wherein x, y, and z are as defined above. In some embodiments, the mixed metal oxide includes CeₓNd_{y}O_{z}, wherein x, y, and z are as defined above. In some embodiments, the mixed metal oxide includes CeₓSm_{y}O_{z}, wherein x, y, and z are as defined above. In some embodiments, the mixed metal oxide includes CeₓTb_{y}O_{z}, wherein x, y, and z are as defined above.

In some embodiments, the mixed metal oxide includes CeₓAl_{y}O_{z}, where x is greater than 0 or from 1 to 10, y is greater than 0 or from 1 to 10, and z is greater than 0 or at least 1. For example, in some embodiments, the mixed metal oxide includes Ce₁Al₉O_{z}, where z is greater than 0 or at least 1. In some embodiments, the mixed metal oxide includes Ce₂Al₈O_{z}, where z is greater than 0 or at least 1. In some embodiments, the mixed metal oxide includes Ce₃Al₇O_{z}, where z is greater than 0 or at least 1. In some embodiments, the mixed metal oxide includes Ce₄Al₆O_{z}, where z is greater than 0 or at least 1. In some embodiments, the mixed metal oxide includes CeAlO_{z}, where z is greater than 0 or at least 1. In some embodiments, the mixed metal oxide includes Ce₆Al₄O_{z}, where z is greater than 0 or at least 1. In some embodiments, the mixed metal oxide includes Ce₇Al₃O_{z}, where z is greater than 0 or at least 1. In some embodiments, the mixed metal oxide includes Ce₈Al₂O_{z}, where z is greater than 0 or at least 1. In some embodiments, the mixed metal oxide includes Ce₉Al₁O_{z}, where z is greater than 0 or at least 1.

In some embodiments, the atomic ratio of the redox component to the acid component is in the range of X:Y, where X is the number of atoms of the redox component and ranges from 0 to 100, and where Y is the number of atoms of the acid component and ranges from 0 to 100. In some embodiments, the atomic ratio is determined by XRF or XRD, among other techniques and/or measurements. In some embodiments, the atomic ratio of Ce to M ranges from 0 to 1 inclusive, or any incremental value or subrange between that range. For example, in some embodiments, the atomic ratio of Ce to M is about 0.1. In some embodiments, the atomic ratio of Ce to M is about 0.2. In some embodiments, the atomic ratio of Ce to M is about 0.3. In some embodiments, the atomic ratio of Ce to M is about 0.4. In some embodiments, the atomic ratio of Ce to M is about 0.5. In some embodiments, the atomic ratio of Ce to M is about 0.6. In some embodiments, the atomic ratio of Ce to M is about 0.7. In some embodiments, the atomic ratio of Ce to M is about 0.8. In some embodiments, the atomic ratio of Ce to M is about 0.9. In some embodiments, the atomic ratio of Ce to M is about 1.0.

In some embodiments, the mixed metal oxide includes one or more of the following: CeO₂—Al₂O₃, TiO₂—SiO₂, TiO₂—Al₂O₃, TiO₂—CdO, TiO₂—Bi₂O₃, TiO₂—Sb₂O₅, TiO₂—SnO₂, TiO₂—ZrO₂, TiO₂—BeO, TiO₂—MgO, TiO₂—CaO, TiO₂—SrO, TiO₂—ZnO, TiO₂—Ga₂O₃, TiO₂—Y₂O₃, TiO₂—La₂O₃, TiO₂—MoO₃, TiO₂—Mn₂O₃, TiO₂—Fe₂O₃, TiO₂—Co₃O₄, TiO₂—WO₃, TiO₂—V₂O₅, TiO₂—Cr₂O₃, TiO₂—ThO₂, TiO₂—Na₂O, TiO₂—BaO, TiO₂—CaO, TiO₂—HfO₂, TiO₂—Li₂O, TiO₂—Nb₂O₅, TiO₂—Ta₂O₅, TiO₂—Gd₂O₃, TiO₂—Lu₂O₃, TiO₂—Yb₂O₃, TiO₂—CeO₂, TiO₂—Sc₂O₃, TiO₂—PbO, TiO₂—NiO, TiO₂—CuO, TiO₂—CoO, TiO₂—B₂O₃, ZrO₂—SiO₂, ZrO₂—Al₂O₃, ZrO₂—SnO, ZrO₂—PbO, ZrO₂—Nb₂O₅, ZrO₂—Ta₂O₅, ZrO₂—Cr₂O₃, ZrO₂—MoO₃, ZrO₂—WO₃, ZrO₂—TiO₂, ZrO₂—HfO₂, TiO₂—SiO₂—Al₂O₃, TiO₂—SiO₂—ZnO, TiO₂—SiO₂—ZrO₂, TiO₂—SiO₂—CuO, TiO₂—SiO₂—MgO, TiO₂—SiO₂—Fe₂O₃, TiO₂—SiO₂—B₂O₃, TiO₂—SiO₂—WO₃, TiO₂—SiO₂—Na₂O, TiO₂—SiO₂—MgO, TiO₂—SiO₂—La₂O₃, TiO₂—SiO₂—Nb₂O₅, TiO₂—SiO₂—Mn₂O₃, TiO₂—SiO₂—Co₃O₄, TiO₂—SiO₂—NiO, TiO₂—SiO₂—PbO, TiO₂—SiO₂—Bi₂O₃, TiO₂—Al₂O₃—ZnO, TiO₂—Al₂O₃—ZrO₂, TiO₂—Al₂O₃—Fe₂O₃, TiO₂—Al₂O₃—WO₃, TiO₂—Al₂O₃—La₂O₃, TiO₂—Al₂O₃—Co₃O₄, ZrO₂—SiO₂—Al₂O₃, ZrO₂—SiO₂—SnO, ZrO₂—SiO₂—Nb₂O₅, ZrO₂—SiO₂—WO₃, ZrO₂—SiO₂—TiO₂, ZrO₂—SiO₂—MoO₃, ZrO₂—SiO₂—HfO₂, ZrO₂—SiO₂—Ta₂O₅, ZrO₂—Al₂O₃—SiO₂, ZrO₂—Al₂O₃—PbO, ZrO₂—Al₂O₃—Nb₂O₅, ZrO₂—Al₂O₃—WO₃, ZrO₂—Al₂O₃—TiO₂, ZrO₂—Al₂O₃—MoO₃, ZrO₂—HfO₂—Al₂O₃, ZrO₂—HfO₂—TiO₂, and the like.

The vanadium and/or vanadia loading of the catalyst composition can range from greater than 0 wt. % to about 80 wt. % inclusive, or any incremental value or subrange between that range. Unless otherwise provided, the wt. % is based on the total weight of the catalyst composition. In some embodiments, the vanadium and/or vanadia loading is about 1 wt. %. In some embodiments, the vanadium and/or vanadia loading is about 3 wt. %. In some embodiments, the vanadium and/or vanadia loading is about 5 wt. %. In some embodiments, the vanadium and/or vanadia loading is about 7 wt. %. In some embodiments, the vanadium and/or vanadia loading is about 9 wt. %. In some embodiments, the vanadium and/or vanadia loading is about 10 wt. %. In some embodiments, the vanadium and/or vanadia loading is about 12 wt. %. In some embodiments, the vanadium and/or vanadia loading is about 14 wt. %. In some embodiments, the vanadium and/or vanadia loading is about 15 wt. %. In some embodiments, the vanadium and/or vanadia loading is about 16 wt. %. In some embodiments, the vanadium and/or vanadia loading is about 18 wt. %. In some embodiments, the vanadium and/or vanadia loading is about 20 wt. %. In some embodiments, the vanadium and/or vanadia loading is about 22 wt. %. In some embodiments, the vanadium and/or vanadia loading is about 24 wt. %. In some embodiments, the vanadium and/or vanadia loading is about 25 wt. %. In some embodiments, the vanadium and/or vanadia loading is about 26 wt. %. In some embodiments, the vanadium and/or vanadia loading is about 28 wt. %. In some embodiments, the vanadium and/or vanadia loading is about 30 wt. %.

In some embodiments, the cerium and/or ceria loading is in the range of about greater than 0 wt. % to about 100 wt. % inclusive, or any incremental value or subrange between that range. Unless otherwise provided, the wt. % is based on the total weight of the catalyst composition. In some embodiments, the cerium and/or ceria loading is about 1 wt. %. In some embodiments, the cerium and/or ceria loading is about 3 wt. %. In some embodiments, the cerium and/or ceria loading is about 5 wt. %. In some embodiments, the cerium and/or ceria loading is about 7 wt. %. In some embodiments, the cerium and/or ceria loading is about 9 wt. %. In some embodiments, the cerium and/or ceria loading is about 10 wt. %. In some embodiments, the cerium and/or ceria loading is about 12 wt. %. In some embodiments, the cerium and/or ceria loading is about 14 wt. %. In some embodiments, the cerium and/or ceria loading is about 15 wt. %. In some embodiments, the cerium and/or ceria loading is about 16 wt. %. In some embodiments, the cerium and/or ceria loading is about 18 wt. %. In some embodiments, the cerium and/or ceria loading is about 20 wt. %. In some embodiments, the cerium and/or ceria loading is about 22 wt. %. In some embodiments, the cerium and/or ceria loading is about 24 wt. %. In some embodiments, the cerium and/or ceria loading is about 25 wt. %. In some embodiments, the cerium and/or ceria loading is about 26 wt. %. In some embodiments, the cerium and/or ceria loading is about 28 wt. %. In some embodiments, the cerium and/or ceria loading is about 30 wt. %.

In some embodiments, the pore size of the mixed metal oxide (e.g., mixed metal oxide support) is at least 0.1 nm, or at least any one of, equal to any one of, or between any two of 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and 35 nm.

The average particle size of the catalyst composition can be in the range of about 1 nm to about 500 nm inclusive, or any incremental value or subrange between that range. For example, in some embodiments, the average particle size of the catalyst composition is about 10 nm, about 20 nm, about 30 nm, about 40 nm, 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, 310 nm, about 320 nm, about 330 nm, about 340 nm, 350 nm, about 360 nm, about 370 nm, about 380 nm, about 390 nm, about 400 nm, about 410 nm, about 420 nm, about 430 nm, about 440 nm, 450 nm, about 460 nm, about 470 nm, about 480 nm, about 490 nm, or about 500 nm.

In some embodiments, the redox and acidic properties may be adjusted via tuning the Ce/Al atomic ratio of the support material and that the content in cerium may improve the oxidative ability, whereas aluminum species may increase the amount of weak acid sites and total acid sites. An excess in cerium may provide more over-oxidative products such as CO₂, CO, and methyl formate. Additionally, the high aluminum-containing catalysts may offer more acidic amount leading to the dehydration product dimethyl ether directly without the oxidation reaction. In some embodiments, a V₂O₅/CeAlOₓ catalyst composition may achieve the highest methanol conversion (62.1%) and DMM selectivity (85.2%). In the long-term test, the V₂O₅/CeAlOₓ catalyst composition served more than 500 h lifetime with only 10% of methanol conversion decay. The ratio of Ce/Al is tuned for the selective reaction of methanol to DMM; the suitable support may enhance the catalytic performance and also result in extending the lifetime of the catalyst.

### Selective Oxidation of Methanol

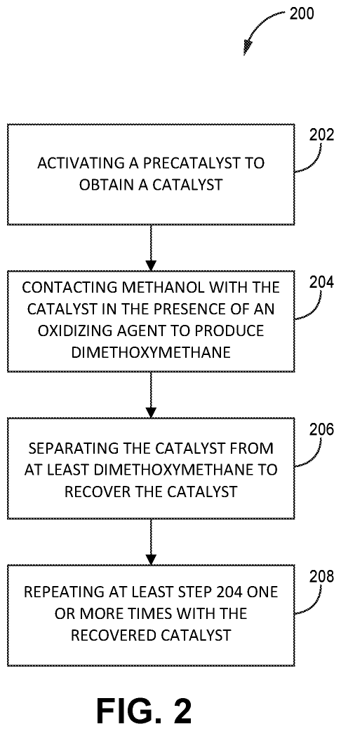

FIG. 2 is a flowchart of a method of forming dimethoxymethane by selective oxidation of methanol in accordance with one or more embodiments of the present invention. As shown in FIG. 2, the method 200 can comprise one or more of the following steps: (a) activating 202 a precatalyst to obtain a catalyst; (b) contacting 204 methanol with the catalyst in the presence of an oxidizing agent to produce dimethoxymethane; (c) separating 206 the catalyst from at least the reaction products to recover the catalyst; and (d) repeating 208 step (b) one or more times with the recovered catalyst. In some embodiments, such as embodiments in which the catalyst is poisoned among other situations, the catalyst may be regenerated and reused. For example, in some embodiments, the catalyst may be regenerated by subjecting the recovered catalyst (such as the catalyst recovered in step (c)) to calcination (e.g., exposure to temperatures of about 300° C., or temperatures of at least 200° C. to about 1000° C., or any incremental value or subrange between that range) and conditions for activating or reactivating the catalyst (e.g., such as activating according to step (a), for example, among others), optionally before proceeding to step (d) in which step (b) may be repeated one or more times.

Step (a) includes activating the precatalyst to obtain the catalyst. The activating can include reducing the precatalysts in H₂, Ar, CO, or a combination thereof for a duration of at least about 1 minute. The activation temperature can range from about 200° C. to about 800° C. inclusive, or any incremental value or subrange between that range. The precatalysts and catalysts can include any of the catalyst compositions disclosed herein. For example, in some embodiments, the precatalyst and/or catalyst includes vanadium oxide supported on a mixed metal oxide, wherein the mixed metal oxide includes cerium and an acid component.

Step (b) includes contacting methanol with the catalyst in the presence of an oxidizing agent to produce dimethoxymethane. The contacting can include bringing the methanol and catalyst composition into physical contact, or immediate or close proximity. For example, in some embodiments, the contacting includes flowing the methanol over the catalyst composition. In some embodiments, the contacting includes passing the methanol over the catalyst composition. In some embodiments, the contacting includes feeding methanol to a reaction vessel including the catalyst composition. In some embodiments, the contacting includes introducing the methanol to a reaction vessel including the catalyst composition. In some embodiments, the contacting includes injecting the methanol into a reaction vessel including the catalyst composition. In some embodiments, the contacting can include exposing the methanol and oxidizing agent to the catalyst composition. In some embodiments, the contacting includes reacting the methanol and oxidizing agent over the catalyst composition. In some embodiments, the catalyst composition is disposed in a reactor or reaction vessel. The reactors/reaction vessels are not particularly limited. Examples of suitable reactors include, but are not limited to, tank reactors and tubular reactors which can be configured to operate as batch, semi-batch, or continuously stirred tank reactors, fluidized bed reactors, or fixed bed reactors.

The contacting can be performed at or over temperatures in the range of about 0° C. to about 500° C. For example, in some embodiments, the contacting is performed at a temperature of about 500° C., about 450° C., about 400° C., about 375° C., about 350° C., about 340° C., about 330° C., about 320° C., about 310° C., about 300° C., about 290° C., about 280° C., about 270° C., about 260° C., about 250° C., about 240° C., about 230° C., about 220° C., about 210° C., about 200° C., about 190° C., about 180° C., about 170° C., about 160° C., about 150° C., about 140° C., about 130° C., about 120° C., about 110° C., about 100° C., about 90° C., about 80° C., about 70° C., about 60° C., about 50° C., about 40° C., about 30° C., about 20° C., about 10° C., about 0° C., or any increment between those temperatures. In some embodiments, the contacting is performed at a temperature of less than about 300° C., less than about 290° C., less than about 280° C., less than about 270° C., less than about 260° C., less than about 250° C., less than about 240° C., less than about 230° C., less than about 220° C., less than about 210° C., or at least about 200° C. In some embodiments, the contacting is performed at a temperature of about 210° C. In some embodiments, the contacting is performed at a temperature of about 190° C. In some embodiments, the contacting is performed at a temperature in the range of about 180° C. to about 220° C.

The contacting can be performed at or over pressures in the range of about 0.1 bar to about 85 bar. In some embodiments, the contacting is performed at or over pressures in the range of 75 to 85 bar, or about 75 to 80 bar, or any incremental value or subrange between that range. In some embodiments, the contacting is performed at about 80 bar (e.g., about 8.1035 MPa). In some embodiments, the contacting is performed at a pressure of about 85 bar, about 84 bar, about 83 bar, about 82 bar, about 81 bar, about 80 bar, about 79 bar, about 78 bar, about 77 bar, about 76 bar, about 75 bar, about 70 bar, about 65 bar, about 60 bar, about 55 bar, about 50 bar, about 45 bar, about 40 bar, about 35 bar, about 30 bar, about 25 bar, about 20 bar, about 15 bar, about 10 bar, about 5 bar, about 1 bar, about 0.9 bar, about 0.8 bar, about 0.7 bar, about 0.6 bar, about 0.5 bar, about 0.4 bar, about 0.3 bar, about 0.2 bar, about 0.1 bar, or any increment between those pressures. In some embodiments, the contacting is performed at a pressure of less than about 22 bar and/or greater than about 25 bar. In some embodiments, the contacting is performed at a pressure of about 50 bar. In some embodiments, the contacting is performed at a pressure of about 30 bar. In some embodiments, the contacting is performed at a pressure of about 20 bar. In some embodiments, the contacting is performed at about atmospheric pressure.

The contact time can range from about 0.001 s to about 10 s (e.g., in fixed-bed reactors). In some embodiments, the contact time is at least 0.001 s, or at least any one of, equal to any one of, or between any two of 0.001, 0.01, 0.1, 0.2, 0.3, 0.4, 0.5, 0.6, 0.7, 0.8, 0.9, 1, 2, 3, 4, 5, 6, 7, 8, 9, and 10 s. The reaction time can range from about 1 min to about 24 hr (e.g., in batch reactors). In some embodiments, the reaction time is about 12 hr. In some embodiments, the reaction time is about 5 hr. In some embodiments, the reaction time is about 6 hr. In some embodiments, the reaction time is about 7 hr. In some embodiments, the reaction time is about 8 hr. In some embodiments, the reaction time is about 9 hr. In some embodiments, the reaction time is about 10 hr. In some embodiments, the reaction time is about 11 hr. In some embodiments, the reaction time is about 13 hr. In some embodiments, the reaction time is about 14 hr. In some embodiments, the reaction time is about 15 hr.

The methanol can be included in a feed stream. In some embodiments, the feed stream includes methanol, optionally the oxidizing agent, and optionally one or more additional chemical species. In some embodiments, the oxidizing agent and methanol are included the same feed stream. In some embodiments, the oxidizing agent is included in a feed stream which is different from the feed stream containing the methanol. In some embodiments, the additional chemical species include an inert gas, such as argon, nitrogen, or helium. In some embodiments, the additional chemical species includes other alcohols. In some embodiments, the additional chemical species includes other hydrocarbons. In some embodiments, the additional chemical species includes one or more of carbon monoxide, hydrogen, carbon dioxide, ammonia, methane, nitrogen, hydrogen sulfide, helium, and water. In some embodiments, the additional chemical species include one or more of C1-C10 hydrocarbons, nitrogen, ammonia, NOₓ, hydrogen, COₓ, and SOₓ, where x is at least 1.

The oxidizing agent can include a compound including oxygen or any other oxygen source. In some embodiments, the oxidizing agent includes air. In some embodiments, the oxidizing agent includes pure oxygen or O₂.

The ratio of methanol to oxidizing agent can be about 1:1 to about 1:10. In some embodiments, the ratio of methanol to oxidizing agent is about 1:1.5. In some embodiments, the ratio of methanol to oxidizing agent is about 1:2. In some embodiments, the ratio of methanol to oxidizing agent is about 1:1. In some embodiments, the ratio of methanol to oxidizing agent is about 1:3. In some embodiments, the ratio of methanol to oxidizing agent is about 1:4. In some embodiments, the ratio of methanol to oxidizing agent is about 1:5. In some embodiments, the ratio of methanol to oxidizing agent is about 1:6. In some embodiments, the ratio of methanol to oxidizing agent is about 1:7. In some embodiments, the ratio of methanol to oxidizing agent is about 1:8. In some embodiments, the ratio of methanol to oxidizing agent is about 1:9. In some embodiments, the ratio of methanol to oxidizing agent is about 1:10.

The weight hourly space velocity is not particularly limited. For example, in some embodiments, the weight hourly space velocity can be about 0.1 h⁻¹ or greater. In some embodiments, the weight hourly space velocity is about 2.4 h⁻¹.

The reaction products can include at least dimethoxymethane. In some embodiments, the reaction products further include formic acid. In some embodiments, the reaction products further include carbon monoxide. In some embodiments, the reaction products further include carbon dioxide. In some embodiments, the reaction products further include methyl formate. In some embodiments, the reaction products further include dimethyl ether. In some embodiments, the reaction products further include formaldehyde. In some embodiments, the reaction products are substantially free of formic acid. In some embodiments, the reaction products are substantially free of carbon monoxide. In some embodiments, the reaction products are substantially free of carbon dioxide. In some embodiments, the reaction products are substantially free of methyl formate. In some embodiments, the reaction products are substantially free of dimethyl ether. In some embodiments, the reaction products are substantially free of formaldehyde.

The methanol conversion can be about 10% or greater. For example, in some embodiments, the methanol conversion is about 65%. In some embodiments, the methanol conversion is about 64%. In some embodiments, the methanol conversion is about 63%. In some embodiments, the methanol conversion is about 62%. In some embodiments, the methanol conversion is about 61%. In some embodiments, the methanol conversion is about 60%. In some embodiments, the methanol conversion is about 59%. In some embodiments, the methanol conversion is about 58%. In some embodiments, the methanol conversion is about 57%. In some embodiments, the methanol conversion is about 56%. In some embodiments, the methanol conversion is about 55%. In some embodiments, the methanol conversion is about 54%. In some embodiments, the methanol conversion is about 53%. In some embodiments, the methanol conversion is about 52%. In some embodiments, the methanol conversion is about 51%. In some embodiments, the methanol conversion is about 50%. In some embodiments, the methanol conversion is about 49%. In some embodiments, the methanol conversion is about 48%. In some embodiments, the methanol conversion is about 47%. In some embodiments, the methanol conversion is about 46%. In some embodiments, the methanol conversion is about 45%. In some embodiments, the methanol conversion is about 44%. In some embodiments, the methanol conversion is about 43%. In some embodiments, the methanol conversion is about 42%. In some embodiments, the methanol conversion is about 41%. In some embodiments, the methanol conversion is about 40%.

The selectivity towards a desired product(s) of the selective oxidation of methanol can be in the range of about 1% to about 90%, or any incremental value or subrange between that range. In some embodiments, the catalyst compositions can be used to produce dimethoxymethane with a selectivity of at least about 86%, at least about 85.9%, at least about 85.8%, at least about 85.7%, at least about 85.6%, at least about 85.5%, at least about 85.4%, at least about 85.3%, at least about 85.2%, at least about 85.1%, at least about 85%, at least about 84.5%, at least about 84%, at least about 80%, or greater, or any increment thereof. In some embodiments, the catalyst compositions can be used to produce dimethoxymethane with a selectivity of 85.6%. In some embodiments, the catalyst compositions can be used to produce dimethoxymethane with a selectivity of about 60%. In some embodiments, the catalyst compositions can be used to produce dimethoxymethane with a selectivity of 50%. In some embodiments, the catalyst compositions can be used to produce dimethoxymethane with a selectivity of 40%. In some embodiments, the catalyst compositions can be used to produce dimethoxymethane with a selectivity of 30%.

In some embodiments, the catalyst compositions can be used to produce dimethyl ether with a selectivity in the range of about 1% to about 85%. In some embodiments, the catalyst compositions can be used to produce dimethyl ether with a selectivity of less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, or less than about 5%. In some embodiments, the catalyst compositions can be used to produce dimethyl ether with a selectivity of about 8.7%. In some embodiments, the catalyst compositions can be used to produce dimethyl ether with a selectivity of about 10.4%. In some embodiments, the catalyst compositions can be used to produce dimethyl ether with a selectivity of about 14.5%. In some embodiments, the catalyst compositions can be used to produce dimethyl ether with a selectivity of about 20.7%.

In some embodiments, the catalyst compositions can be used to produce methyl formate with a selectivity in the range of about 1% to about 50%. In some embodiments, the catalyst compositions can be used to produce methyl formate with a selectivity of less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, or less than about 5%. In some embodiments, the catalyst compositions can be used to produce methyl formate with a selectivity of about 5%. In some embodiments, the catalyst compositions can be used to produce methyl formate with a selectivity of about 3.1%. In some embodiments, the catalyst compositions can be used to produce methyl formate with a selectivity of about 7.6%. In some embodiments, the catalyst compositions can be used to produce methyl formate with a selectivity of about 17.8%. In some embodiments, the catalyst compositions can be used to produce methyl formate with a selectivity of about 12.6%.

The catalyst compositions can have a long lifetime. For example, in some embodiments, the catalyst composition produces dimethoxymethane with a selectivity that varies no more than about ±10% for a duration ranging from about 0 hr to about 500 hr. In some embodiments, the catalyst composition produces dimethoxymethane with a selectivity that varies no more than about ±15% for a duration ranging from about 0 hr to about 500 hr. In some embodiments, the catalyst composition produces dimethoxymethane with a selectivity that varies no more than about ±20% for a duration ranging from about 0 hr to about 500 hr. In some embodiments, methanol conversion various by no more than about ±5% for a duration ranging from about 0 hr to about 500 hr. In some embodiments, methanol conversion various by no more than about ±10% for a duration ranging from about 0 hr to about 500 hr. In some embodiments, methanol conversion various by no more than about ±15% for a duration ranging from about 0 hr to about 500 hr.

Step (c) includes separating the catalyst composition from at least the reaction products to recover the catalyst composition and step (d) includes repeating step (b) one or more times with the recovered catalyst composition. The catalyst composition can be easily separated from reactants and/or reaction products. In some embodiments, the catalyst composition is removed from the reactor or reaction vessel and optionally regenerated or reactivated (e.g., in accordance with step (a)). Once separated and optionally regenerated or reactivated, the recovered catalyst composition can be reused one or more times in step (b) to produce additional dimethoxymethane.

### Catalyst Synthesis

Dimethoxymethane selectivity and methanol conversion can be significantly improved by modifying vanadium-based catalysts with at least two metal oxides. For example, vanadium-based catalysts can be modified with CeO₂ and Al₂O₃ to obtain V₂O₅/CeₓAl_{y}O_{z} catalysts. High Ce-containing catalysts can lead to over-oxidative products such as carbon dioxide and methyl formate. High Al-containing catalysts can lead to the dehydration product dimethyl ether directly without the oxidation reaction by virtue of the presence of additional acid sites. Accordingly, the Ce and Al content can be balanced to optimize the performance of the catalyst.

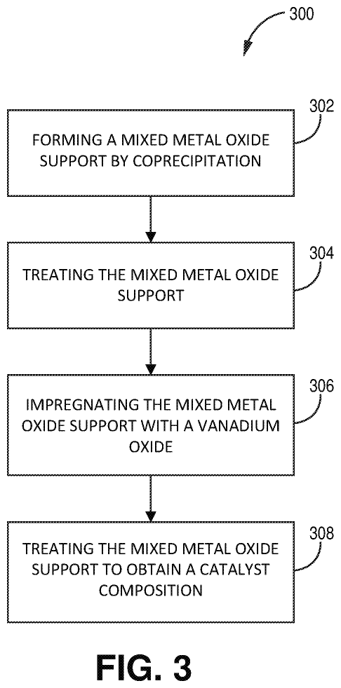

FIG. 3 is a flowchart of a method of synthesizing catalyst compositions for selective oxidation of methanol to dimethoxymethane, according to one or more embodiments of the invention. As shown in FIG. 3, the method of synthesizing catalyst compositions 300 may include one or more of the following steps: (a) forming 302 a mixed metal oxide support by coprecipitation; (b) treating 304 the mixed metal oxide support; (c) impregnating 306 the mixed metal oxide support with a vanadium oxide; and (d) treating 308 the mixed metal oxide support to obtain a catalyst composition. In step (a), formation of the mixed metal oxide support by coprecipitation may include contacting one or more of a precursor for a redox component and a precursor for an acid component in a solution or solvent. In some embodiments, the coprecipitation involves adding K₂CO₃ dropwise under vigorous stirring at about 70° C. for at least 1 hr (e.g., for about 3 hr). In step (b), treatment of the mixed metal oxide support may include one or more of filtering, washing (e.g., with water), drying (e.g., under heating), and calcined at a calcine temperature (e.g., a temperature of about 300° C.). In some embodiments, the mixed metal oxide support is calcined at about 300° C. to convert M(OH)ₓ species into MOₓ species. In step (c) impregnation of the mixed metal oxide support with vanadium oxide may include contacting the mixed metal oxide support with a precursor of vanadium oxide, such as NH₄VO₃. In some embodiments, the impregnation may proceed under heating (e.g., at a temperature of about 50-70° C.), optionally under stirring. In step (d), treatment of the mixed metal oxide support (e.g., the impregnated mixed metal oxide support) may include drying (e.g, under heating at for example about 110° C.) and calcined at about 300° C. for at least 1 hr (e.g., for about 4 hr).

### Example 1

### Synthesis of Catalysts

The Ce—Al mixed oxide support was prepared by co-precipitation, certain amount (atomic ratio of Ce/Al=1/9, 3/7, 1/1, 7/3, and 9/1) of Ce(NO₃)₃·6H₂O (Sigma-Aldrich, ≥99%) and Al(NO₃)₃·9H₂O (Sigma-Aldrich, >99%) was dissolved in 100 mL D.I. water to which 0.6 M of a K₂CO₃ (Sigma-Aldrich, ≥99%) solution was added, drop-by-drop, under vigorous stirring at 70° C. for 3 h. The white-yellow co-precipitated solid was then filtered, washed with D.I. water, and dried overnight at 110° C., finally calcined to 300° C., for 4 h, in order to convert M(OH)ₓ into MOₓ. These catalysts are labeled as CeₓAl_{y}O where the x/y ratio is the atomic ratio of Ce/Al.

Ce/Al supported vanadium catalyst was prepared using the wet impregnation method, and the support materials (Al₂O₃-γ (Sigma-Aldrich, ≥99%), CeO₂ (Sigma-Aldrich, ≥99%), and Ce—Al mixed oxides) were impregnated with a NH₄VO₃ (Sigma-Aldrich, ≥99%) solution (0.25 g of NH₄VO₃ and 5 mL of H₂O_{2(aq)} (Sigma-Aldrich, 30%)), at 60° C., and stirred for 3 h. After impregnation, the samples were dried overnight, at 110° C., and subsequently calcined to 300° C. for 4 h. These catalysts label as V₂O₅/CeₓAl_{y}O (where the x/y ratio is the atomic ratio of Ce/Al).

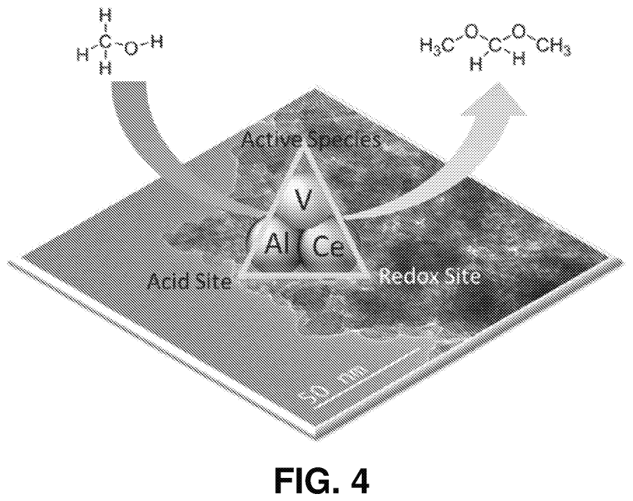

As illustrated in the examples, V₂O₅/CeAlOₓ exhibited strong catalyst performance, achieving methanol conversion of about 61%, high dimethoxymethane selectivity of about 85%, and long working lifetime of over 500 hr. FIG. 4 is a schematic diagram of a Ce—Al mixed metal oxides supported vanadium catalyst for selective oxidation of methanol to dimethoxymethane in accordance with one or more embodiments of the present invention.

### Example 2

### V₂O₅/Al₂O₃Catalyst

The catalytic performance of V₂O₅/Al₂O₃ in the selective oxidation of methanol to dimethoxymethane was evaluated. The reaction conditions included a reaction temperature of about 200° C., reaction time of about 12 hr, and a WHSH_{MeOH} of about 2.37 h⁻¹. The carrier gas included nitrogen (N₂) and was fed to the reactor at a volumetric flow rate of about 11 mL/min. The molar ratio of N₂ to O₂ to MeOH (n_{N2}:n_{O2}:n_{MeOH}) was about 2.24:1.48:1. The catalyst achieved a methanol conversion of about 52.7, produced dimethoxymethane with a selectivity of 14.4%, produced dimethyl ether with a selectivity of 81.1%, and produced methyl formate with a selectivity of 3.1%.

### Example 3

### V₂O₅/Ce₁Al₉Oₓ-co-300

The catalytic performance of V₂O₅/Ce₁Al₉Oₓ-co-300 in the selective oxidation of methanol to dimethoxymethane was evaluated. The reaction conditions included a reaction temperature of about 200° C., reaction time of about 12 hr, and a WHSH_{MeOH} of about 2.37 h⁻¹. The carrier gas included nitrogen (N₂) and was fed to the reactor at a volumetric flow rate of about 11 mL/min. The molar ratio of N₂ to O₂ to MeOH (n_{N2}:n_{O2}:n_{MeOH}) was about 2.24:1.48:1. The catalyst achieved a methanol conversion of about 44.9%, produced dimethoxymethane with a selectivity of 18.4%, produced dimethyl ether with a selectivity of 64.7%, and produced methyl formate with a selectivity of 12.6%.

### Example 4

### V₂O₅/Ce₃Al₇Oₓ-co-300

The catalytic performance of V₂O₅/Ce₃Al₇Oₓ-co-300 in the selective oxidation of methanol to dimethoxymethane was evaluated. The reaction conditions included a reaction temperature of about 200° C., reaction time of about 12 hr, and a WHSH_{MeOH} of about 2.37 h⁻¹. The carrier gas included nitrogen (N₂) and was fed to the reactor at a volumetric flow rate of about 11 mL/min. The molar ratio of N₂ to O₂ to MeOH (n_{N2}:n_{O2}:n_{MeOH}) was about 2.24:1.48:1. The catalyst achieved a methanol conversion of about 59.6%, produced dimethoxymethane with a selectivity of 60.6%, produced dimethyl ether with a selectivity of 20.7%, and produced methyl formate with a selectivity of 17.8.

### Example 5

### V₂O₅/CeAlOₓ-co-300

The catalytic performance of V₂O₅/CeAlOₓ-co-300 in the selective oxidation of methanol to dimethoxymethane was evaluated. The reaction conditions included a reaction temperature of about 200° C., reaction time of about 12 hr, and a WHSH_{MeOH} of about 2.37 h⁻¹. The carrier gas included nitrogen (N₂) and was fed to the reactor at a volumetric flow rate of about 11 mL/min. The molar ratio of N₂ to O₂ to MeOH (n_{N2}:n_{O2}:n_{MeOH}) was about 2.24:1.48:1. The catalyst achieved a methanol conversion of about 62.1%, produced dimethoxymethane with a selectivity of 85.6%, produced dimethyl ether with a selectivity of 8.7%, and produced methyl formate with a selectivity of 5.0%.

### Example 6

### V₂O₅/Ce₇Al₃Oₓ-co-300

The catalytic performance of V₂O₅/Ce₇Al₃Oₓ-co-300 in the selective oxidation of methanol to dimethoxymethane was evaluated. The reaction conditions included a reaction temperature of about 200° C., reaction time of about 12 hr, and a WHSH_{MeOH} of about 2.37 h⁻¹. The carrier gas included nitrogen (N₂) and was fed to the reactor at a volumetric flow rate of about 11 mL/min. The molar ratio of N₂ to O₂ to MeOH (n_{N2}:n_{O2}:n_{MeOH}) was about 2.24:1.48:1. The catalyst achieved a methanol conversion of about 61.2%, produced dimethoxymethane with a selectivity of 42.9%, produced dimethyl ether with a selectivity of 10.4%, and produced methyl formate with a selectivity of 42.6%.

### Example 7

### V₂O₅/Ce₉Al₁Oₓ-co-300

The catalytic performance of V₂O₅/Ce₉Al₁Oₓ-co-300 in the selective oxidation of methanol to dimethoxymethane was evaluated. The reaction conditions included a reaction temperature of about 200° C., reaction time of about 12 hr, and a WHSH_{MeOH} of about 2.37 h⁻¹. The carrier gas included nitrogen (N₂) and was fed to the reactor at a volumetric flow rate of about 11 mL/min. The molar ratio of N₂ to O₂ to MeOH (n_{N2}:n_{O2}:n_{MeOH}) was about 2.24:1.48:1. The catalyst achieved a methanol conversion of about 46.8%, produced dimethoxymethane with a selectivity of 34.3%, produced dimethyl ether with a selectivity of 14.5%, and produced methyl formate with a selectivity of 49.9%.

### Example 8

### V₂O₅/CeO₂

The catalytic performance of V₂O₅/CeO₂ in the selective oxidation of methanol to dimethoxymethane was evaluated. The reaction conditions included a reaction temperature of about 200° C., reaction time of about 12 hr, and a WHSH_{MeOH} of about 2.37 h⁻. The carrier gas included nitrogen (N₂) and was fed to the reactor at a volumetric flow rate of about 11 mL/min. The molar ratio of N₂ to O₂ to MeOH (n_{N2}:n_{O2}:n_{MeOH}) was about 2.24:1.48:1. The catalyst achieved a methanol conversion of about 11.6%, produced dimethoxymethane with a selectivity of <1%, produced dimethyl ether with a selectivity of <1%, produced methyl formate with a selectivity of 7.6%, produced carbon monoxide with a selectivity of about 21%, and produced carbon dioxide with a selectivity of about 75.6%.

### Example 9

### Ce/Al Atomic Ratio

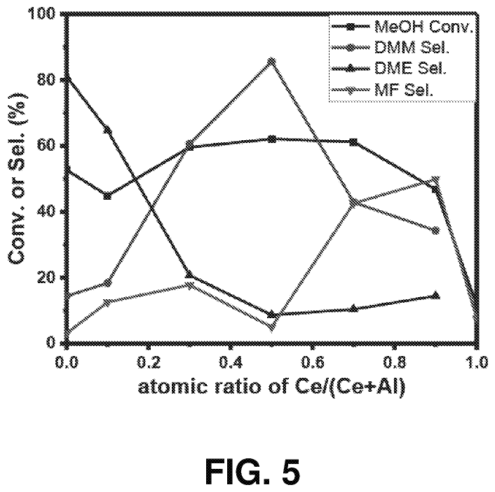

FIG. 5 is a graphical view illustrating the effect of Ce/Al ratios on conversion or selectivity in accordance with one or more embodiments of the present invention.

### Example 10

### Characterization of Catalysts

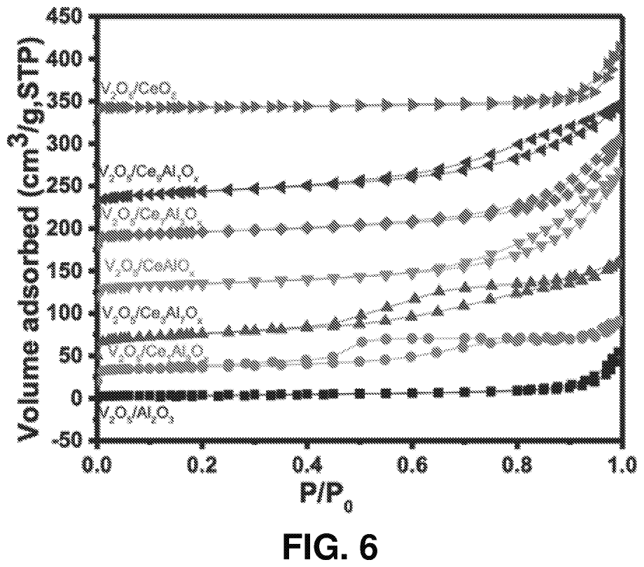

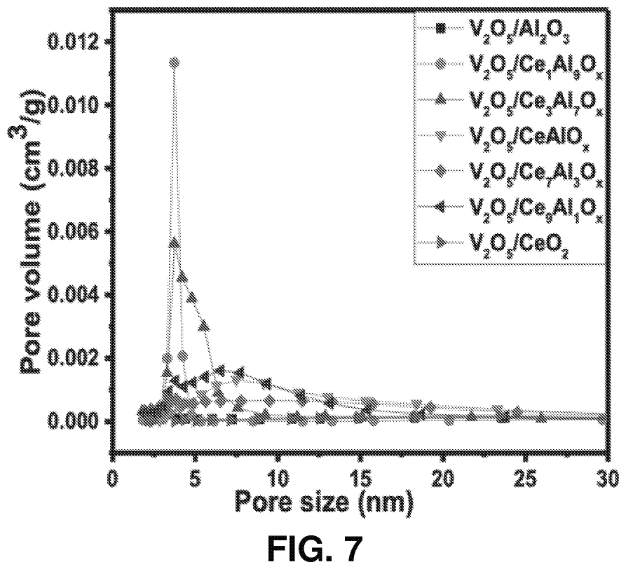

Textural properties of the catalysts were determined by N₂ adsorption-desorption analysis. Results are presented in Table 1. Ce—Al mixed metal oxide supported catalysts all exhibited a higher BET surface area than the mono-metal oxide supported catalyst; the pore size increased with increasing amounts of cerium. The mesoporosity decreased as the amount of cerium increased (FIG. 6 and FIG. 7), due to the fact that aluminum oxide is a mesoporous material. Real Ce/Al atomic ratios were determined using XRF measurements, these values were similar to the theoretical ratio values, for all Ce—Al mixed metal oxide supported catalysts (Table 1).

TABLE 1
Texture Properties

| | SBET | | | Particle size (nm)^{a} | | | |
|---|---|---|---|---|---|---|---|
| Catalyst | (m²/g) | D_{P} (nm) | V_{P} (cm³/g) | V₂O₅ | Al₂O₃ | CeO₂ | Ce/Al^{b} |
| V₂O₅/Al₂O₃ | 13.7 | 21.5 | 0.08 | 27.9 | 37.8 | — | — |
| V₂O₅/Ce₁Al₉Oₓ | 57.6 | 5.1 | 0.11 | 23.1 | — | — | 0.16 |
| V₂O₅/Ce₃Al₇Oₓ | 92.3 | 5.8 | 0.17 | 36.1 | — | 3.5 | 0.44 |
| V₂O₅/CeAlOₓ | 55.5 | 12.3 | 0.23 | 38.0 | — | 4.2 | 0.93 |
| V₂O₅/Ce₇Al₃Oₓ | 56.4 | 12.6 | 0.20 | 26.0 | — | 3.5 | 2.33 |
| V₂O₅/Ce₉Al₁Oₓ | 83.7 | 8.8 | 0.19 | 32.6 | — | 4.1 | 17.89 |
| V₂O₅/CeO₂ | 12.2 | 25.3 | 0.11 | 35.0 | — | 29.3 | — |

^{a}Caculated by Scherrer-eqation
^{b}Atomic ratio determined by XRF

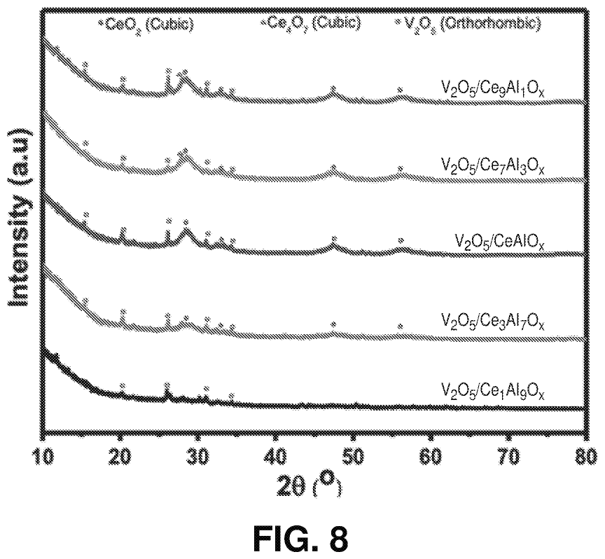

The XRD pattern (FIG. 8) showed strong V₂O₅ peaks (2θ=15.4°, 20.3°, 26.3°, and 31.1°, JCPDS card no. 01-072-0433) for all catalysts, and intense CeO₂ peaks (2θ=28.5°, 33.0°, 47.5°, and 56.2°, JCPDS card no. 00-041-1426) for all catalysts except for the V₂O₅/Ce₁Al₉Oₓ catalyst. This result was explained by the fact this particular catalyst only contained a small amount of cerium. The Ce₄O₇ peak (2θ=27.9°, JCPDS card no. 65-7999) appeared for catalysts rich in cerium (V₂O₅/Ce₇Al₃Oₓ and V₂O₅/Ce₉Al₁Oₓ). Aluminum can stabilize CeO₂ and inhibit the formation of a Ce₄O₇ crystal structure, leading to the formation (by low cerium-containing catalysts) of CeO₂ only, and without the Ce₄O₇ crystal structure. XRD patterns also showed that the Al₂O₃ crystal structure was missing from all catalysts, due to a calcination temperature (300° C.) too low to be able to form the crystalline aluminum oxide. The particle size of each metal oxide was calculated using the Scherrer-equation (Table 1). Calculations showed that the V₂O₅/CeAlOₓ catalyst had the largest V₂O₅ and CeO₂ particles, and possessed the best crystalline structure.

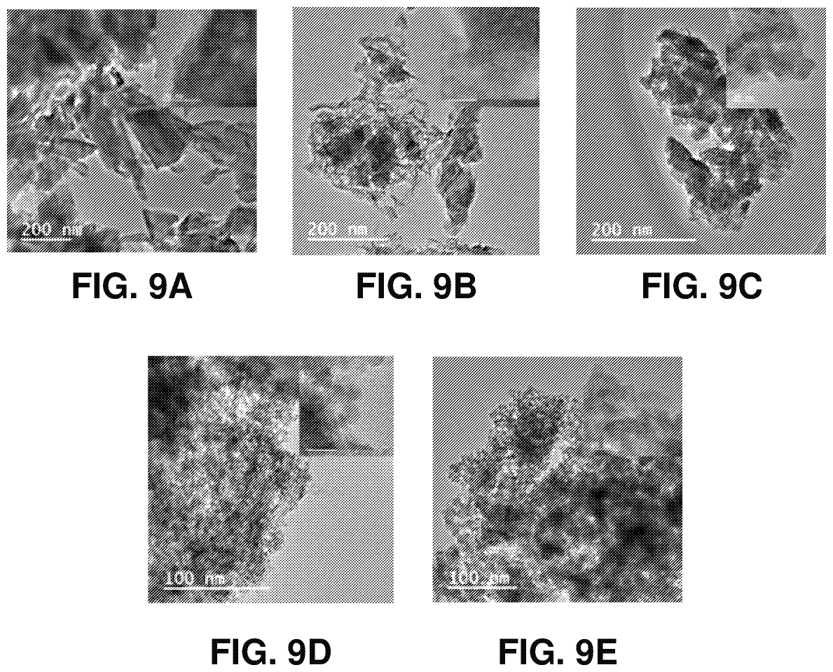

TEM images (FIGS. 9A-9E) showed that the morphology of the catalyst changed as the Ce/Al atomic ratio varied, evolving from a flak shape (more similar to aluminum oxide) to a nanocluster as the amount of cerium increased. High-resolution fringes pattern confirmed that a high Ce/Al ratio led to a better crystallinity, consistently with XRD measurements.

TABLE 2
Acidic and Redox Properties

| | NH₃ uptake | | | H₂ uptake | | | |
|---|---|---|---|---|---|---|---|
| | (mmol/g_{cat}) | | | (mmol/g_{cat}) | | | |
| Catalyst | Weak | Strong | Total | P1 | P2 | P3 | Total |
| V₂O₅/Ce₁Al₉Oₓ-co-300 | 2.3 | 0.51 | 2.8 | | 2.1 | 1.7 | 3.8 |
| V₂O₅/Ce₃Al₇Oₓ- co-300 | 2.2 | 0.63 | 2.8 | — | 2.2 | 1.2 | 3.4 |
| V₂O₅/CeAlOₓ-co-300 | 1.4 | 0.81 | 2.3 | — | 2.3 | 1.7 | 4.0 |
| V₂O₅/Ce₇Al₃Oₓ-co-300 | 1.1 | 1.0 | 2.1 | 0.73 | 1.2 | 1.9 | 3.8 |
| V₂O₅/Ce₉Al₁Oₓ-co-300 | 0.63 | 1.1 | 1.7 | 0.92 | 0.75 | 2.4 | 4.0 |

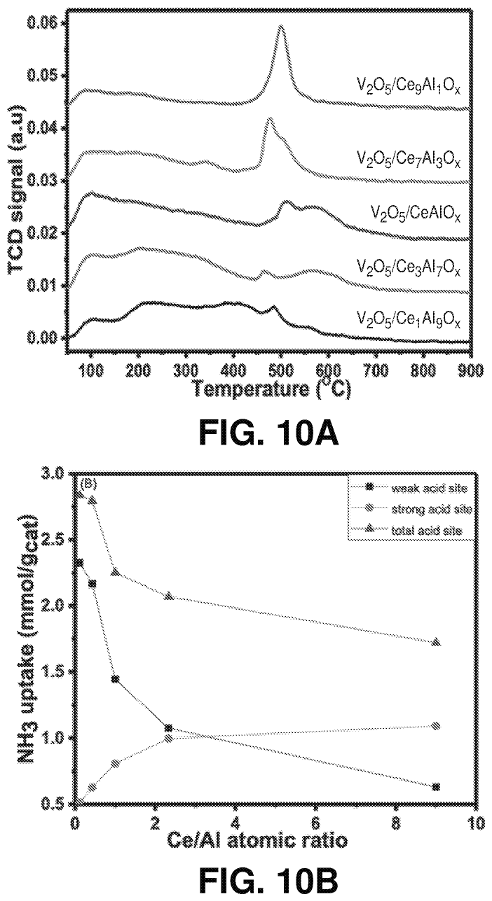

Surface acidity was determined by using NH₃-TPD (FIGS. 10A-10B and Table 2). Results showed that the amount of weak acid sites (50˜450° C.) decreased (NH₃ uptake from 2.3 to 0.63 mmol/gcat) as the amount of cerium amount increased, leading to the decrease in the total amount of acid as the content in aluminum decreased (NH₃ uptake from 2.8 to 1.7 mmol/gcat). Additionally, the amount of strong acid sites (450˜700° C.) increased (NH₃ uptake from 0.51 to 1.1 mmol/gcat) as the cerium amount increased was observed; the relation between the Ce/Al atomic ratio and the amount of acid sites is presented in FIG. 10B. These results indicated that the amount of weak acid sites and the amount of total acid were related to the aluminum content and that strong acid sites relied on the presence of cerium. Although catalysts containing low amounts of cerium offered fewer strong acid sites, the T. of low cerium-contained catalysts was presented at higher temperature region which means that the aluminum serves stronger acidity than cerium.

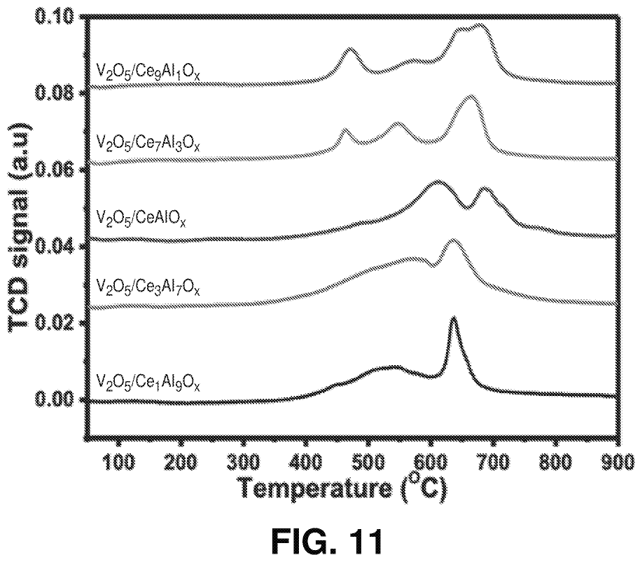

Redox properties and H₂ consumption were measured by using H₂-TPR (FIG. 11 and Table 3-3-2). Catalysts rich in cerium (V₂O₅/Ce₇Al₃Oₓ, and V₂O₅/Ce₉Al₁Oₓ) showed a sharp reduction of the peak (around 460° C.) associated with the reduction of surface Ce₄+ ions, due to the high content in cerium. According to previous reports, the high-temperature region (500˜750° C.) is related to the reduction of vanadium species. In this region, all catalysts exhibit two reduction peaks due to the two reactions V₂O₅→VO₂ and VO₂→V₂O₃, except for V₂O₅/Ce₉Al₁Oₓ, which offers a splitting peak around 760° C. This peak corresponds to the transformation from CeVO₄ to CeVO₃. Because the excess Ce⁴⁺ was anchored by vanadium species, the reducibility of the cerium was reduced and the reduction peak shifted to a higher temperature.

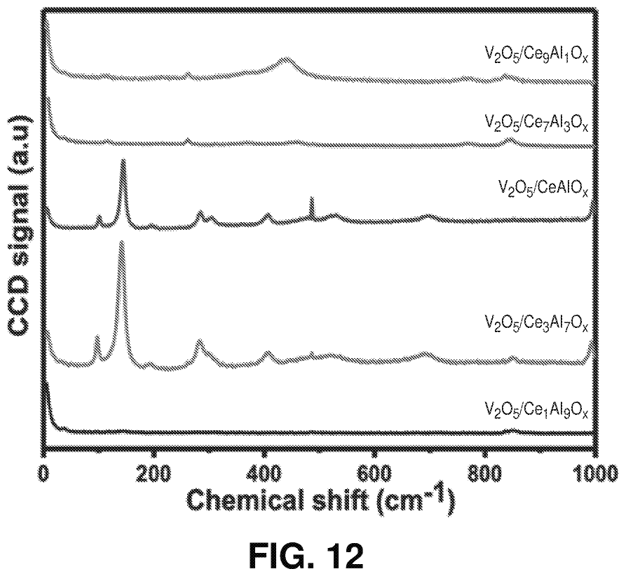

Raman spectroscopy enabled us to analyze the mixed oxides material provided evidence of the amorphous species that could not be detected by XRD. FIG. 12 shows that the catalyst containing a small amount of cerium did not exhibit the CeO₂ peaks at 260 cm⁻¹ and 460 cm⁻¹. Additionally, only V₂O₅/Ce₉Al₁Oₓ showed a strong peak at both 770 cm⁻¹ and 845 cm⁻¹, these two peaks were associated with CeVO₄ species, confirming the results from H₂-TPR. However, V₂O₅/Ce₃Al₇Oₓ and V₂O₅/CeAlOₓ both gave two strong peaks around 140 cm⁻¹ and 990 cm⁻¹, which could be assigned to V₂O₅ and the surface V═O, respectively. These results revealed the critical role played by the Ce/Al ratio (3/7 and 1/1) with respect to the formation of a stable V₂O₅.

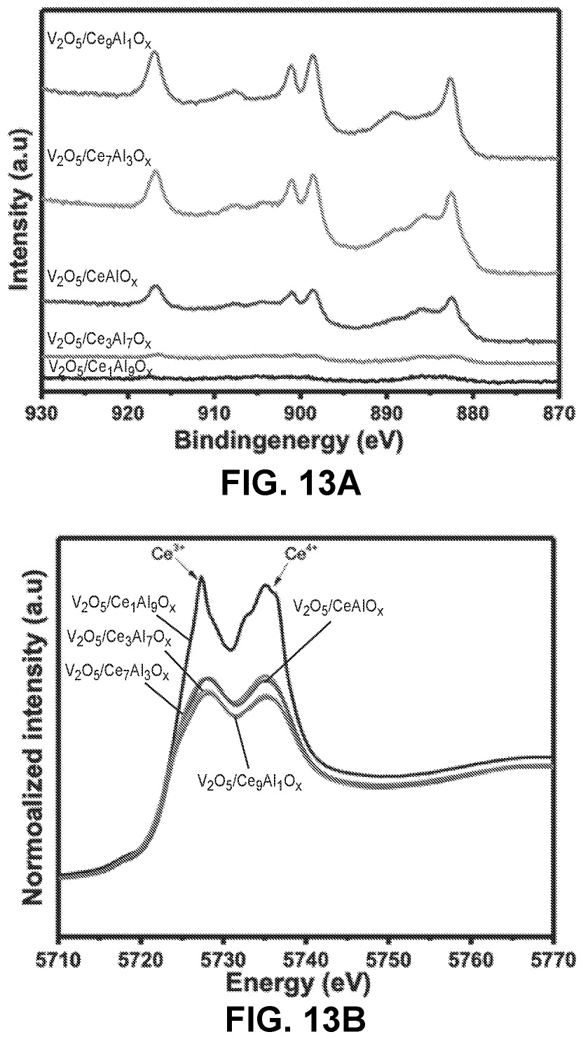

Results from X-ray adsorption measurements showing Ce3d spectra are presented in FIG. 13A. Catalysts with a low content in cerium did not exhibit any intense peak; however, their intensity increased with increasing amounts of cerium. The bonding energy of Ce3d showed two peak sets, Ce³⁺ (904 and 885 eV) and Ce⁴⁺ (917, 907, 901, 898, 889 and 882 eV). The catalyst with the highest content in cerium (V₂O₅/Ce₉Al₁Oₓ) only offered the Ce⁴⁺ oxidation state, whereas the other two catalysts showed both Ce³⁺ and Ce⁴⁺ oxidation states. Due to the detection limits of XPS, only the Ce L3-edge XANES spectra (FIG. 13B) were presented, indicated that the Ce⁴⁺/Ce³⁺ ratio increased as the amount of cerium increased.

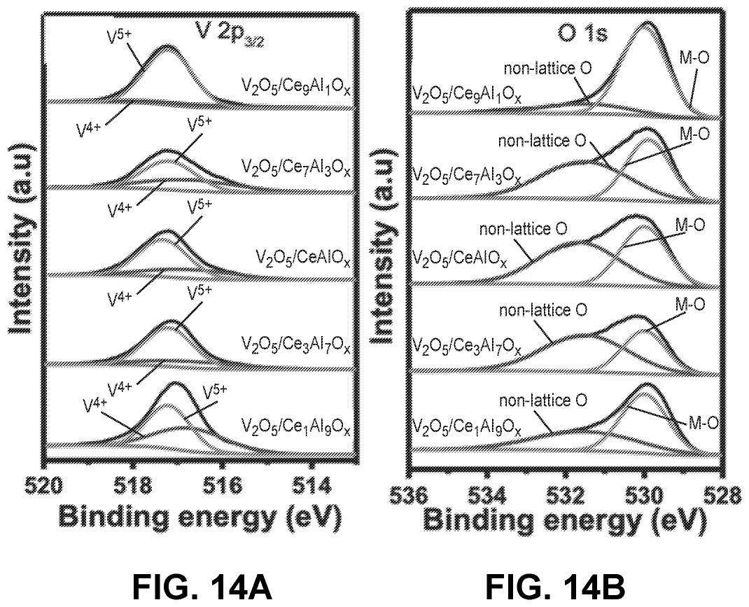

These results also provided evidence that the V₂O₅/Ce₉Al₁Oₓ offered the highest oxidation ability. FIGS. 14A-14B shows the peak fittings of V2p (FIG. 14A) and O1s (FIG. 14B).

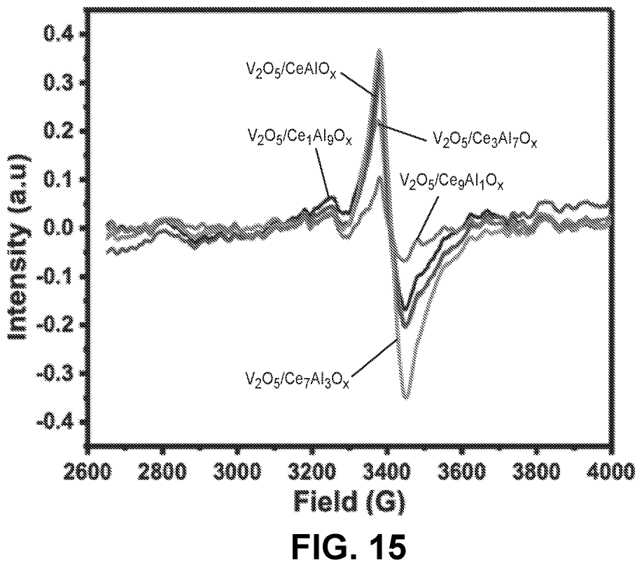

All catalysts contained two oxidation states of the vanadium: V⁵⁺ and V⁴⁺. The V₂O₅/Ce₉Al₁Oₓ catalyst was found to have the lowest content in V⁴⁺ (13.1%, Table 3) which was confirmed by EPR measurements (spectra shown in FIG. 15). A higher content in V⁵⁺ provided a stronger oxidative ability, similarly to Ce⁴⁺. It was hypothesized that the lattice oxygen played a more important role than the non-lattice oxygen in the oxidation reaction. V₂O₅/Ce₉Al₁Oₓ catalyst provided the highest amount of lattice oxygen (82.5%, Table. 3), meaning that this catalyst offered the highest oxidative ability. This fining further confirmed the results obtained from Ce3d and V2p spectroscopy measurements.

TABLE 3
Peak Fitting Analysis for V2p and O1s XPS

| | V 2p (eV) | | O 1s (eV) | | V⁴⁺/(V⁴⁺ + V⁵⁺) | (O_{lat.}/(O_{lat.} O_{non-lat.}) |
|---|---|---|---|---|---|---|
| Catalyst | V⁴⁺ | V⁵⁺ | O_{lat.} | O_{non-lat.} | (%) | (%) |
| V₂O₅/Ce₁Al₉Oₓ | 531.6 | 531.6 | 531.6 | 531.6 | 531.6 | 531.6 |
| V₂O₅/Ce₃Al₇Oₓ | 531.6 | 531.6 | 531.6 | 531.6 | 531.6 | 531.6 |
| V₂O₅/CeAlOₓ | 531.6 | 531.6 | 531.6 | 531.6 | 531.6 | 531.6 |
| V₂O₅/Ce₇Al₃Oₓ | 531.6 | 531.6 | 531.6 | 531.6 | $31.6 | 531.6 |
| V₂O₅/Ce₉Al₁Oₓ | 531.6 | 531.6 | 531.6 | 531.6 | 531.6 | 531.6 |

### EXAMPLE

### Catalytic Performance

The catalytic performance of each catalyst is listed in Table 4. The selective oxidation of methanol to DMM included the oxidation of methanol to formaldehyde followed by the acetalization reaction (dehydration reaction) between methanol and formaldehyde. The first oxidation step was catalyzed by the redox site, and the second acetalization reaction was catalyzed by the acidic site of the catalyst. However, the redox and acidic property had an impact on catalysis: if the catalyst provides a very strong oxidative ability, the over-oxidative product (such as methyl formate, CO, and CO₂) will form. On the other hand, if the acidic property is too strong, the main product will be the dehydration product (such as dimethyl ether) without any oxidation reaction. In this example, a series of supported vanadium catalysts with different Ce/Al atomic ratios are provided. The aluminum-supported V₂O₅ catalysts (V₂O₅/Al₂O₃) give the dehydration product dimethyl ether, as the major product; cerium oxide-supported V₂O₅ catalysts (V₂O₅/CeO₂) show a higher selectivity towards the over-oxidative product, such as CO, CO₂, and/or methyl formate. However, the Ce—Al mixed metal oxide-supported V₂O₅ catalysts all provided higher DMM selectivity. Compared with the two mono-metal-supported catalysts, a result revealing that the mixing of cerium and aluminum can improve catalytic performance.

The V₂O₅/CeAlOₓ catalyst provided the best catalytic performance (62.1% of methanol conversion and 85.2% DMM selectivity). When the aluminum was the major component (like in V₂O₅/Ce₁Al₉Oₓ and V₂O₅/Ce₃Al₇Oₓ), the high amount of acid sites (FIGS. 10A-10B) affected the reaction to form the dehydration-only product (dimethyl ether) as the simple aluminum-supported catalyst.

TABLE 4
Catalytic Performance

| | MeOH | | | | |
|---|---|---|---|---|---|
| | Conv. | Selectivity (%) | | | |
| Catalyst | (%) | DMM | DME | MF | T (° C.) |
| V₂O₅/Al₂O₃ | 52.7 | 14.4 | 81.1 | 3.1 | 200 |
| V₂O₅/Ce₁Al₉Oₓ | 44.9 | 18.4 | 64.7 | 12.6 | 200 |
| V₂O₅/Ce₃Al₇Oₓ | 59.6 | 60.6 | 20.7 | 17.8 | 200 |
| V₂O₅/CeAlOₓ | 62.1 | 85.6 | 8.7 | 5.0 | 200 |
| V₂O₅/Ce₇Al₃Oₓ | 61.2 | 42.9 | 10.4 | 42.6 | 200 |
| V₂O₅/Ce₉Al₁Oₓ | 46.8 | 34.3 | 14.5 | 49.9 | 200 |
| V₂O₅/CeO₂* | 11.6 | <1 | <1 | 7.6 | 200 |

Reaction condition: WHSV_{MeOH} = 2.37 h-1, reaction time = 12 h, carrier gas (N2) = 11 mL/min, n_{N2}:n_{O2}:n_{MeOH} = 2.24:1.48:1
*CO Sel. = 21%, CO₂ Sel. = 75%

Results presented in Table 4 also show that the selectivity of the methyl formate increased with the content in cerium, due to its higher oxidative ability. Because the catalysts containing a high amount of cerium (V₂O₅/Ce₇Al₃Oₓ, and V₂O₅/Ce₉Al₁Oₓ) formed more easily the CeVO₄ species (FIG. 11, and FIG. 12), this CeVO₄ can offer a higher oxidative ability, as well as a higher amount of Ce⁴⁺, V⁵⁺, with the lattice oxygen becoming more significant as the content in cerium increases (FIGS. 13A-13B and FIGS. 14A-14B).

### Example 10

### Catalyst Stability

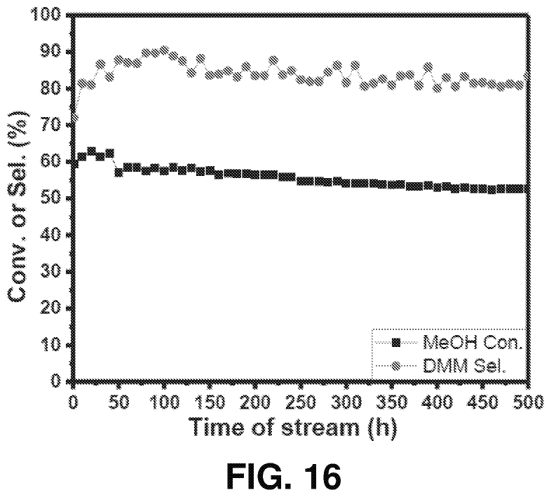

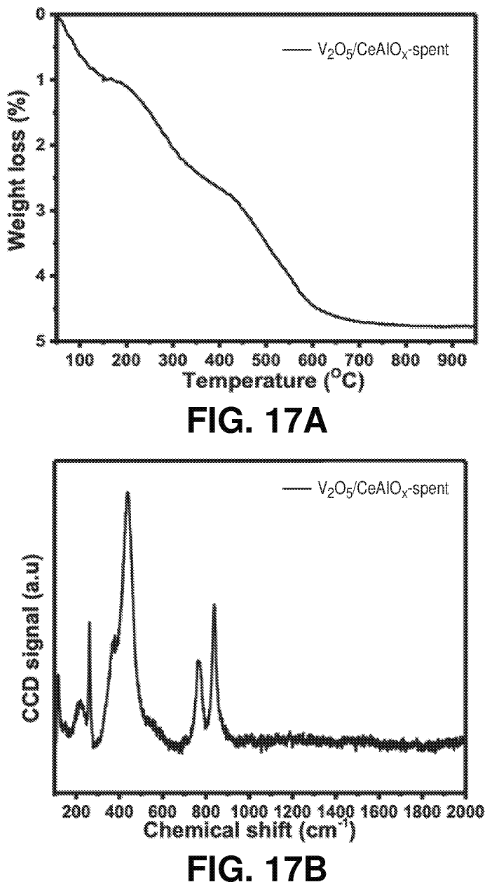

FIG. 16 is a graphic view illustrating the stability of a Ce—Al mixed metal oxides supported vanadium catalyst in accordance with one or more embodiments of the present invention. V₂O₅/CeAlOₓ exhibits great stability and high performance (FIG. 16) when subjected to long-term tests. We find it to have a lifetime of over 500 h lifetime, losing less than 10% in methanol conversion (62.4 to 52.6%), and without the decay of DMM selectivity. The result from TGA measurement (FIG. 17A) shows that the spent catalyst only produces 5% of coke, while the Raman spectra (FIG. 17B) shows no D band (1350 cm⁻¹) or G band (1580 cm⁻¹) signal. These results further confirm that our catalyst produces a very low amount of coke while offering to provide high stability to the catalyst.

1. A catalyst composition for use in the formation of dimethoxymethane by selective oxidation of methanol, the catalyst composition comprising:
a mixed metal oxide support impregnated with a vanadium oxide, wherein the mixed metal oxide support includes at least a redox component and an acid component.

2. The catalyst composition according to claim 1, wherein the redox component includes cerium.

3. The catalyst composition of claim 1, wherein the mixed metal oxide support is represented by the following chemical formula:
CeₓM_{y}O_{z}
wherein M is the acid component, x is from 1 to 9, y is from 1 to 9, z is at least 1.

4. The catalyst composition according to claim 3, wherein the acid component includes at least one of the following: Al, Cr, Co, Fe, Mn, Mo, Nb, Sb, Sc, Y, La, Ti, Zr, Hf, Ta, W, Re, Ru, Rh, Ir, Nᵢ, Pd, Pt, Cu, Ag, Au, Zn, B, Ga, In, Pb, P, As, Bi, Se, Pr, Nd, Sm, and Tb.

5. The catalyst composition of claim 1, wherein the acid component includes aluminum.

6. The catalyst composition of claim 1, wherein the vanadium oxide includes at least V₂O₅.

7. The catalyst composition of claim 1, wherein the catalyst composition is represented by the following chemical formula:
V₂O₅/CeₓM_{y}O_{z}
wherein M is the acid component, x is from 1 to 9, y is from 1 to 9, z is at least 1.

8. The catalyst composition according to claim 7, wherein the catalyst composition includes V₂O₅/CeAlO_{z}, where z is at least 1.

9. The catalyst composition according to claim 7, wherein the catalyst composition includes at least one of the following: V₂O₅/CeAlO_{z}, V₂O₅/Ce₁Al₉O_{z}, V₂O₅/Ce₃Al₇O_{z}, V₂O₅/Ce₇Al₃O_{z}, V₂O₅/Ce₉Al₁O_{z}, wherein z is at least 1.

10. The catalyst composition according to claim 1, wherein the redox component includes cerium, the acid component includes aluminum, and the atomic ratio of Ce to Al is from 1:9 to 9:1.

11. The catalyst composition according to claim 10, wherein the vanadium oxide includes V₂O₅.

12. A method of forming dimethoxymethane by selective oxidative of methanol, comprising:
contacting methanol with a catalyst composition in the presence of an oxidizing agent to produce dimethoxymethane, wherein the catalyst composition includes a mixed metal oxide support impregnated with a vanadium oxide and wherein the mixed metal oxide support includes a redox component and an acid component.

13. The method according to claim 12, wherein the redox component includes cerium, the acid component includes aluminum, and the vanadium oxide includes V₂O₅.

14. The method of claim 12, wherein the oxidizing agent is air.

15. The method of claim 12, wherein the contacting step is performed at reaction temperatures in the range of about 180° C. to about 220° C.

16. The method of claim 12, wherein the contacting step is performed for a reaction time of about 12 h.

17. The method of claim 12, wherein the weight hourly space velocity is at least about 2.00 h⁻¹.

18. The method of claim 12, wherein the catalyst composition produces dimethoxymethane with a selectivity of at least about 85%.

19. The method of claim 12, wherein methanol conversion is at least about 60%.

20. The method of claim 12, wherein the reaction products include less than about 10% of dimethyl ether and/or methyl formate.
